(19) United States
(12) Patent Application Publication
(10) Pub. No.: US 2022/0241334 A1
(43) Pub. Date: Aug. 4, 2022

(54) COMPOSITIONS AND METHODS FOR TREATING MYOCARDIUM WITH MESENCHYMAL STEM CELL SCAFFOLD

(71) Applicants: United States Government As Represented By The Department of Veterans Affairs, Washington, DC (US); Regents Of The University of Minnesota, Minneapolis, MN (US)

(72) Inventors: Laura Leigh Hocum Stone, Minneapolis., MN (US); Rosemary Frances Kelly, Minneapolis, MN (US); Edward Oakley McFalls, Minneapolis, MN (US); Christin Wright, Minneapolis, MN (US)

(21) Appl. No.: 17/623,805

(22) PCT Filed: Jul. 2, 2020
(86) PCT No.: PCT/US2020/040612

§ 371 Date: Dec. 29, 2021

Related U.S. Application Data
(60) Provisional application No. 62/870,165, filed on Jul. 3, 2019.

(51) Int. Cl. A61K 35/17; A61L 27/18; A61L 27/58; A61P 9/04; A61P 9/10

(52) U.S. Cl. CPC A61K 35/17; A61L 27/18; A61L 2430/20; A61P 9/04; A61P 9/10; A61L 27/58

(57) ABSTRACT

Disclosed are methods of increasing cardiac function in a subject's myocardium comprising: administering a scaffold to the subject's myocardium, wherein the scaffold comprises mesenchymal stem cells (MSCs). Disclosed are methods of restoring the mitochondrial proteome in a subject's myocardium comprising: administering a scaffold to the subject's myocardium, wherein the scaffold comprises mesenchymal stem cells. Disclosed are methods of treating hibernating myocardium comprising combining surgical revascularization with attaching a scaffold comprising MSCs to myocardium.

### CROSS REFERENCE TO RELATED APPLICATIONS

This application claims the benefit of Provisional Application No. 62/870,165, filed on Jul. 3, 2020, the content of which is hereby incorporated by reference herein in its entirety.

### STATEMENT REGARDING FEDERALLY SPONSORED RESEARCH

This invention was made with government support under VA Merit Review 101 BX000760 awarded by United States Government as represented by the Department of Veterans Affairs. The government has certain rights in the invention.

### BACKGROUND

“Hibernating myocardium” (HM) refers to chronically ischemic heart tissue in the distribution of a severe coronary artery stenosis, and is characterized by decreased regional blood flow and function at rest and the presence of contractile reserve (2). Although the tissue is viable, HM in patients with advanced 3-vessel disease does not recover completely with coronary artery bypass graft surgery (CABG), suggesting that alternate therapies are needed, potentially at the time of CABG.

The energetic demand of heart tissue is critically dependent on optimal mitochondrial function, which is facilitated by efficient electron transport within the inner mitochondrial membrane. Within isolated mitochondria from a swine model of HM, expression of key proteins within the individual complexes is distinctly different from post-ischemic-reperfused heart tissue was shown (9). Within HM, the expression of electron transport chain (ETC) proteins remain depressed, despite successful revascularization with CABG. These observations suggest that the process of mitochondrial dynamism is incomplete with CABG alone and that recovery of the mitochondrial proteome remains inadequate. In fact, the recovery of heart mitochondria in response to acute and chronic myocardial ischemia is dependent upon a dynamic balance involving fission, fusion and autophagy to preserve mitochondrial energetics and optimize contractile function.

In order to fully recover myocardial energetics, mitochondria must fuse and coalesce. To no surprise, cardiac mitochondria from HM appear smaller and more variable in size and might explain why contractile reserve does not return to normal following CABG. The proteins responsible for mitochondrial fusion are transcriptionally regulated by PGC1α, a master-switch of mitochondrial biogenesis, which was found to remain depressed in HM, even following CABG. To improve mitochondrial function, by virtue of enhanced PGC1α signaling, administration of mesenchymal stem cells (MSCs) in a rat model of diabetic cardiomyopathy improved cardiac function. Similarly, in a mouse model of acute kidney injury, stem cell treatment resulted in enhanced mitochondrial biogenesis by virtue of increased PGC1α. Adult-derived stem cells are appealing therapeutics as they are easy to obtain, easy to expand ex vivo, and lack ethical conflicts. Accordingly, in this study, therapeutic potential of an MSC cardiac patch as an adjunct therapy at the time of CABG to provide more complete recovery of HM hearts, specifically as a mechanism to restore the mitochondrial proteome, was determined.

### BRIEF SUMMARY

Disclosed are methods of increasing cardiac function in a subject's myocardium comprising: administering a scaffold to the subject's myocardium, wherein the scaffold comprises mesenchymal stem cells (MSCs).

Disclosed are methods of restoring the mitochondrial proteome in a subject's myocardium comprising: administering a scaffold to the subject's myocardium, wherein the scaffold comprises mesenchymal stem cells

Disclosed are methods of treating hibernating myocardium comprising combining surgical revascularization with attaching a scaffold comprising MSCs to myocardium.

Disclosed are methods of increasing mitochondrial biogenesis in ischemic myocardium comprising administering a scaffold comprising MSCs to the ischemic myocardium.

Disclosed are methods of increasing PGC1α in ischemic myocardium comprising administering a scaffold comprising MSCs to the ischemic myocardium.

In some aspects of the disclosed methods, the scaffold is administered to the subject in combination with coronary artery bypass graft surgery.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an experimental design to test MSC cardiac patch in a swine model of hibernating myocardium (HM). Juvenile swine are implanted with a rigid plastic constrictor that creates a gradual, non-occluding stenosis as the animal grows over 12 weeks. This creates a model of single-vessel HM characterized by decreased blood flow and decreased cardiac function with preserved tissue viability. At 12 weeks, animals undergo coronary artery bypass graft (CABG) surgery with simultaneous treatment of either a mesenchymal stem cell (MSC) cardiac patch or a sham patch without cells. Four weeks following this treatment, cardiac function is measured by cardiac MRI and the animal is sacrificed for proteomic analysis of the cardiac tissue.

FIGS. 2A-2D show the characterization of mesenchymal stem cell (MSC) cardiac patch. A) To confirm MSC phenotype, allogeneic swine bone marrow-derived MSCs were assessed by flow cytometry and positively express MSC surface markers CD90 and CD105 but do not express the hematopoietic marker CD45. B) Light microscopy of the MSC patch seven days after MSC seeding, just prior to implantation, shows MSCs forming a monolayer within the polyglactin mesh (dark strands). C) Two cardiac patches consisting of 3.5 cm polyglactin mesh circles loaded with MSCs in situ on epicardium during CABG. D) Hematoxylin and eosin staining of the patch (patch area indicated by the black dotted line) on the epicardium four weeks following implantation does not indicate any inflammatory response within the host myocardium. Minor inflammation is present at the suture sites (black arrows) as indicated by dark staining of infiltrating immune cells.

FIGS. 3A and 3B show treatment with mesenchymal (MSC) cardiac patch improves regional cardiac function under dobutamine stress as compared to sham patch. Measurements of regional cardiac function using cardiac MRI show that A) Regional left ventricle function as measured by wall thickening % is not significantly improved at rest with MSC patch treatment (n=6) as compared to sham (n=6). B) Regional wall thickening % is significantly improved under increased work (dobutamine infusion) following treatment with the MSC patch (n=6) as compared to sham animals (n=6), p<0.05 as measured by Mann-Whitney test. Horizontal bars indicate mean±SD.

FIGS. 4A and 4B show treatment with mesenchymal (MSC) cardiac patch does not provide additional improvements to blood flow as compared to sham patch. Regional blood flow was measured by infusion of fluorescently labeled microspheres at rest and under increased work with dobutamine infusion. A) At rest, there is no significant change in regional blood flow across experimental conditions. B) Under dobutamine infusion, both groups receiving coronary artery bypass graft (CABG) (n=6/group) show an improvement in regional blood flow as compared to hibernating myocardium (HM) (n=8) (p<0.05), but adjunct therapy with the MSC patch does not significantly improve perfusion, as measured by one-way ANOVA with Tukey's test. Horizontal bars indicate mean±SD.

FIGS. 5A-5E show treatment with mesenchymal (MSC) cardiac patch increased average cardiac mitochondrial size and density as compared to sham patch. Using transmission electron microscopy (TEM), representative tissue samples from the hibernating myocardium (HM) region of animals treated with the MSC patch (n=4) and sham patch (n=4) were used to count mitochondrial density (#/40 μM\hat{ }2) and size. A) Mitochondrial size (p<0.0001, Mann-Whitney) and (B) number (p<0.05, unpaired t-test) are increased following MSC patch treatment as compared to animals receiving a sham patch. Horizontal bars indicate mean±SD. TEM images (8800× magnification) of untreated HM show a dysregulation of mitochondrial size and alignment (D) as compared to healthy myocardium (C). Treatment with coronary artery bypass graft (CABG)+MSC patch shows mitochondrial morphology more consistent with that of healthy myocardium (E).

FIGS. 6A-6C show treatment with mesenchymal stem cell (MSC) cardiac patch increases expression of mitochondrial proteins as compared to sham patch. Western blots show significant increases in protein levels of A) active, nuclear-bound PGC1α, a driver of mitochondrial biogenesis (p<0.005, Mann-Whitney), B) ATP synthase, complex V of the electron transport chain (ETC) (p<0.05, unpaired t-test), and C) succinate dehydrogenase, complex II of the ETC (p<0.05, unpaired t-test) (n=4/group). Horizontal bars indicate mean±SD. All protein level measurements were normalized to a total protein stain to correct for loading variability.

FIGS. 7A-7C show tandem mass tag (TMT) proteomic pathway analysis of mitochondrial fractions indicates upregulation of mitochondrial function pathways following treatment with mesenchymal stem cell (MSC) patch as compared to sham. Isolated mitochondrial fractions from the hibernating myocardium (HM) region of animals treated with MSC patch (n=6) and sham patch (n=6) were profiled by TMT proteomics. Peptides with positive fold changes in response to MSC treatment were processed using protein pathway analysis (PANTHER database) to determine: A) Enriched biological processes and B) enriched subset of metabolic processes as a result of MSC treatment. C) list of proteins in the cellular metabolic pathway enriched by MSC treatment.

FIGS. 8A and 8B show mesenchymal stem cell (MSC) cardiac patch treatment resulted in increased anti-oxidant signaling as compared to sham patch. Tandem mass tag (TMT) proteomics of isolated mitochondria from the hibernating myocardium (HM) region show A) an increase in antioxidant signaling in coronary artery bypass graft (CABG)+MSC treated hearts (n=6) as measured by fold-change compared to sham treated hearts (n=6), B) most notably, glutathione peroxidase shows a 33-fold increase with MSC cardiac patch treatment as compared to hearts treated by sham patch.

FIG. 9 shows a proposed mechanism of action of MSC therapy for HM.

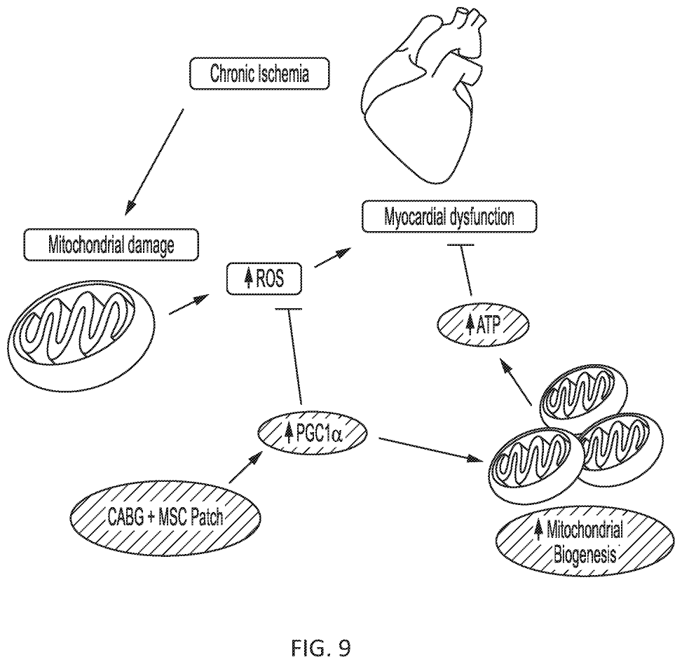

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a mesenchymal stem cell” includes a plurality of such mesenchymal stem cells, reference to “the scaffold” is a reference to one or more scaffolds and equivalents thereof known to those skilled in the art, and so forth.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

As used herein, “administering a scaffold” refers to attaching a scaffold to myocardium. Thus, in some aspects, administering a scaffold can be a surgical procedure. In some aspects, attaching can be contacting the scaffold to myocardium and allowing for self-adhesion. In some aspects, attaching can be physically adhering the scaffold to myocardium. For example, staples, sutures, glue or surgical clips can be used.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Increasing Cardiac Function

Disclosed are methods of increasing cardiac function in a subject's myocardium comprising: administering a scaffold to the subject's myocardium, wherein the scaffold comprises MSCs.

In some aspects, the subject's myocardium has undergone an abnormality. In some aspects, the abnormality can be a decrease in oxygen or other nutrients, decrease in blood flow, or change in contractile function to the myocardium. In some aspects, the subject's myocardium is hibernating myocardium. Hibernating myocardium can be a state wherein a portion or all of the myocardium exhibits abnormalities of contractile function.

In some aspects, the scaffold is administered to the subject in combination with coronary artery bypass graft surgery. Thus, in some aspects, the subject has undergone coronary artery bypass graft (CABG) surgery prior to receiving a scaffold comprising MSCs. In some aspects, a subject receives CABG and a scaffold comprising MSCs simultaneously (i.e., during the same surgical procedure or up to one week following CABG).

Disclosed are methods of increasing cardiac function in a subject's myocardium comprising: administering a scaffold to the subject's myocardium, wherein the scaffold comprises MSCs supported by polygalactin mesh, wherein ATP synthase expression is increased in mitochondria present in the myocardium. In some aspects, the addition of MSCs to the myocardium can increase the expression of mitochondrial electron transport chain proteins within resident mitochondria. In some aspects, the increase in expression of mitochondrial electron transport chain proteins is due to PGC1α signaling. In some aspects, PGC1α expression is increased in mitochondria present in the myocardium. In some aspects, succinate dehydrogenase expression is increased in mitochondria present in the myocardium.

In some aspect, a subject's myocardium must still be viable. Thus, the myocardium can have undergone an abnormality or be diseased, but it must still be viable in order to receive any of the disclosed scaffolds.

1. Scaffold

Disclosed herein are scaffolds comprising mesenchymal stem cells (MSCs). Any of the scaffolds described herein can be used in the methods disclosed herein.

In some aspects, the scaffold can be a surgical mesh or any other bioabsorbable woven material. For example, the surgical mesh can be a polygalactin-based mesh, or any other absorbable woven suture material made primarily of polyglycolic acid. In some aspects, the polygalactin-based mesh is Vicryl®.

In some aspects, MSCs can be seeded onto the scaffold prior to administration of the scaffold to the subject. In some aspects, the seeded cells are cultured on the scaffold for 2, 3, 4, 5, 6, 7, 8, 9, 10, 11, 12, 13, 14, 15, 16, 17, 18, 19, 20, 21, 22, 23, 24, 25, 26, 27, 28, 29, or 30 days prior to administration to a subject. In some aspects, the scaffold comprises a monolayer or multi-layer biofilm of MSCs. For example, a monolayer or multi-layer biofilm of MSCs can form on the scaffold during the culturing of the seeded cells. In some aspects, the scaffold comprises 4-10×10⁶ MSCs. In some aspects, the scaffold can be cultured with 4×10⁶ MSCs prior to attaching it to the myocardium.

In some aspects, the disclosed scaffold can be administered to a subject. In some aspects, administering the scaffold comprises attaching the scaffold to the myocardium via suture, surgical clips, staples, glue or any surgically applicable adhesive. In some aspects, the scaffold is attached to healthy myocardium surrounding an area of abnormal myocardium. In some aspects, the scaffold is attached to abnormal myocardium. In some aspects, the scaffold is attached to a combination of healthy and abnormal myocardium. Abnormal myocardium is tissue that has undergone an abnormality such as, but not limited to, a decrease in oxygen or other nutrients, decrease in blood flow, decrease in ejection fraction, increase in myocardial stiffness, or change in contractile function to the myocardium.

In some aspects, the MSCs are CD90+, CD105+, CD73+ and CD45−, CD34−, CD11b−, HLA Class II−.

### C. Methods of Restoring the Mitochondrial Proteome

Disclosed are methods of restoring the mitochondrial proteome in a subject's myocardium comprising: administering a scaffold to the subject's myocardium, wherein the scaffold comprises mesenchymal stem cells.

In some aspects, the subject's myocardium has undergone an abnormality. In some aspects, the abnormality can be a decrease in oxygen or other nutrients, decrease in blood flow, or change in contractile function to the myocardium. In some aspects, the subject's myocardium is hibernating myocardium. Hibernating myocardium can be a state wherein a portion or all of the myocardium exhibits abnormalities of contractile function.

In some aspects, the scaffold is administered to the subject in combination with coronary artery bypass graft surgery. Thus, in some aspects, the subject has undergone CABG surgery prior to receiving a scaffold comprising MSCs. In some aspects, a subject receives CABG and a scaffold comprising MSCs simultaneously (i.e., during the same surgical procedure or up to one week following CABG).

Disclosed are methods of restoring the mitochondrial proteome in a subject's myocardium comprising: administering a scaffold to the subject's myocardium, wherein the scaffold comprises MSCs, wherein ATP synthase expression is increased in mitochondria present in the myocardium. In some aspects, the addition of MSCs to the myocardium can increase the expression of mitochondrial electron transport chain proteins. In some aspects, the increase in expression of mitochondrial electron transport chain proteins is due to PGC1α signaling. In some aspects, PGC1α expression is increased in mitochondria present in the myocardium. In some aspects, succinate dehydrogenase expression is increased in mitochondria present in the myocardium.

In some aspect, a subject's myocardium must still be viable. Thus, the myocardium can have undergone an abnormality or be diseased, but it must still be viable in order to receive any of the disclosed scaffolds.

### D. Methods of Treating Hibernating Myocardium

Disclosed are methods of treating hibernating myocardium comprising combining surgical revascularization with attaching a scaffold comprising MSCs to myocardium. Hibernating myocardium can be a state wherein a portion or all of the myocardium exhibits abnormalities of contractile function.

In some aspects, surgical revascularization is coronary artery bypass graft surgery or percutaneous coronary intervention. In some aspects, the subject has undergone CABG surgery prior to receiving a scaffold comprising MSCs. In some aspects, a subject receives CABG and a scaffold comprising MSCs simultaneously (i.e., during the same surgical procedure or up to one week following CABG).

Disclosed are methods of treating hibernating myocardium in a subject's myocardium comprising: administering a scaffold to the subject's myocardium, wherein the scaffold comprises MSCs, wherein ATP synthase expression is increased in mitochondria present in the myocardium. In some aspects, the addition of MSCs to the myocardium can increase the expression of mitochondrial electron transport chain proteins. In some aspects, the increase in expression of mitochondrial electron transport chain proteins is due to PGC1α signaling. In some aspects, PGC1α expression is increased in mitochondria present in the myocardium. In some aspects, succinate dehydrogenase expression is increased in mitochondria present in the myocardium.

In some aspect, a subject's myocardium must still be viable. Thus, the myocardium can have undergone an abnormality or be diseased, but it must still be viable in order to receive any of the disclosed scaffolds.

### E. Methods of Increasing Mitochondrial Biogenesis in Ischemic Myocardium

Disclosed are methods of increasing mitochondrial biogenesis in ischemic myocardium comprising administering a scaffold comprising MSCs to the ischemic myocardium.

In some aspects, the scaffold is administered to the ischemic myocardium in combination with coronary artery bypass graft surgery. Thus, in some aspects, a subject having ischemic myocardium has undergone CABG surgery prior to receiving a scaffold comprising MSCs. In some aspects, a subject receives CABG and a scaffold comprising MSCs simultaneously (i.e., during the same surgical procedure or up to one week following CABG).

Disclosed are methods of increasing mitochondrial biogenesis in ischemic myocardium comprising: administering a scaffold to the ischemic myocardium, wherein the scaffold comprises MSCs, wherein ATP synthase expression is increased in mitochondria present in the myocardium. In some aspects, the addition of MSCs to the myocardium can increase the expression of mitochondrial electron transport chain proteins. In some aspects, the increase in expression of mitochondrial electron transport chain proteins is due to PGC1α signaling. In some aspects, PGC1α expression is increased in mitochondria present in the myocardium. In some aspects, succinate dehydrogenase expression is increased in mitochondria present in the myocardium.

In some aspect, the ischemic myocardium must still be viable. Thus, the myocardium can have undergone an ischemic event, but it must still be viable in order to receive any of the disclosed scaffolds.

### F. Methods of Increasing PGC1α in Ischemic Myocardium

Disclosed are methods of increasing PGC1α in ischemic myocardium comprising administering a scaffold comprising MSCs to the ischemic myocardium. In some aspects, an increase in PGC1α comprises an increase in PGC1α gene expression and/or protein expression.

In some aspects, the ischemic myocardium is hibernating myocardium.

In some aspects, the scaffold is administered to ischemic myocardium in combination with coronary artery bypass graft surgery. Thus, in some aspects, the subject has undergone coronary artery bypass graft (CABG) surgery prior to receiving a scaffold comprising MSCs. In some aspects, a subject receives CABG and a scaffold comprising MSCs simultaneously (i.e., during the same surgical procedure or up to one week following CABG.

Disclosed are methods of increasing PGC1α in ischemic myocardium comprising: administering a scaffold to ischemic myocardium, wherein the scaffold comprises MSCs, wherein ATP synthase expression is increased in mitochondria present in the myocardium. In some aspects, the addition of MSCs to the myocardium can increase the expression of mitochondrial electron transport chain proteins. In some aspects, the increase in expression of mitochondrial electron transport chain proteins is due to PGC1α signaling. In some aspects, PGC1α expression is increased in mitochondria present in the myocardium. In some aspects, succinate dehydrogenase expression is increased in mitochondria present in the myocardium.

In some aspect, the ischemic myocardium must still be viable. Thus, the myocardium can have undergone an ischemic event, but it must still be viable in order to receive any of the disclosed scaffolds.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for preparing any of the disclosed scaffolds, the kit comprising a bioabsorbable woven material and MSCs. The kits also can contain instructions for how to use the scaffold.

### EXAMPLES

### A. Example 1

### 1. Methods

i. Animal Use

Group sizes were determined by a priori power analysis using G*Power software. Using the means from the data for the primary endpoint, an effect size of 7.9 and an alpha of 0.05 was determined, resulting in 6 animals per group.

ii. Study Design

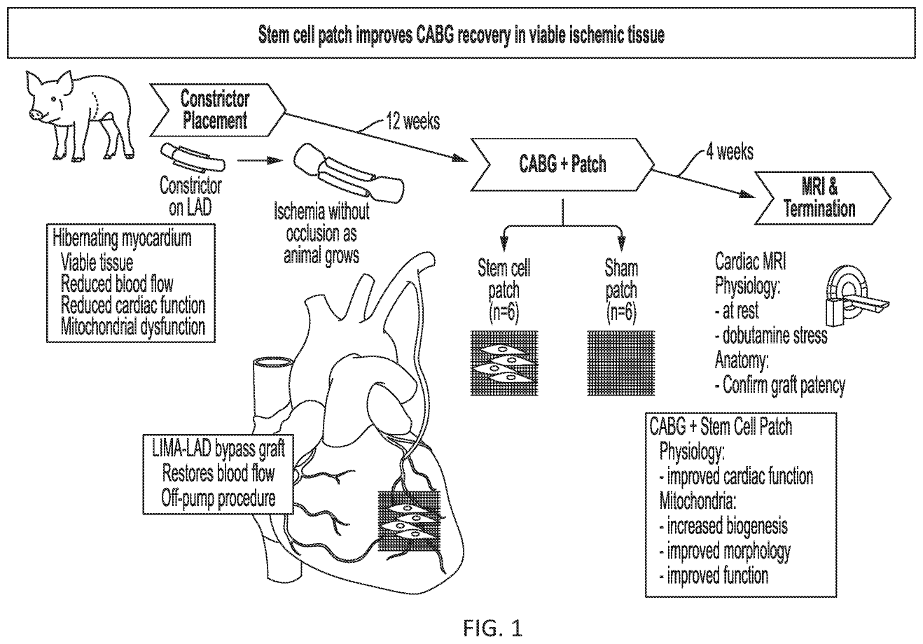

Briefly, 12 animals were subjected to initial instrumentation with a rigid constrictor on the LAD to induce the HM phenotype over 12 weeks. At 12 weeks, the animals were randomly assigned to two treatment groups—either CABG with a stem cell patch (CABG+MSC), or CABG with a sham patch without cells (CABG+sham). Animals were allowed to recover for 4 weeks, after which function was assessed with cardiac MRI and the animal was sacrificed for assessment of gross anatomy and proteomic studies (FIG. 1).

iii. Swine Model of Hibernating Myocardium

HM was induced as previously described. Briefly, juvenile female swine (8-10 kg, n=12) were sedated with telazol (4 mg/kg; IM) and xylazine (2 mg/kg; IM), intubated and anesthetized with isoflurane (2%). A plastic c-shaped constrictor (internal diameter 1.5 mm) was placed via left thoracotomy on the LAD proximal to the first diagonal artery without occluding the artery and secured with non-absorbable sutures. Animals then grow for 12 weeks to establish the HM phenotype in the LAD distribution.

iv. Coronary Artery Bypass+Experimental Treatment

At 12 weeks, pigs undergo CABG under general anesthesia as noted above. CABG was performed via sternotomy with the LIMA pedicle graft dissected free from the chest wall. Lidocaine (1 mg/kg) and heparin (200 units/kg) was administered. The LAD distal to the site of stenosis was exposed, and arteriotomy made to prepare for anastomosis as well as to confirm arterial flow distal to stenosis, confirming the hibernation model of partial occlusion. Using off-pump technique and coronary shunt, LIMA-LAD anastomosis was performed. Immediately following anastomosis, a patch containing either MSCs (n=6) or sham patch without cells (n=6) was sutured onto the hibernating region of the heart using polypropylene suture. The porcine sternotomy was then closed.

v. Cardiac MRI

Cardiac MRI (CMR) imaging was performed under general anesthesia, obtaining short-axis images both at rest and after dobutamine injection (5 μg/kg/min) to simulate cardiac stress. These images were then analyzed for left ventricular (LV) contractile volume and wall strain. Delayed-enhancement images were also obtained after injecting a gadolinium-based contrast agent to rule out infarction.

vi. Terminal Surgery Procedure

Four weeks after CABG, a second cardiac MRI was performed. Pigs then underwent terminal surgery, at which point LAD vessel diameter was measured using coronary dilators at several sites: pre-constrictor, post-constrictor, and LIMA anastomosis. For all animals, the stenosis was no larger than 1.0 mm. Patency of the LIMA-LAD graft was also confirmed using coronary dilators at this time, and all animals in the study had a patent graft that measured within 2.5-3.5 mm. Regional perfusion was assessed at the termination study using fluorescent microspheres (details below).

vii. Regional Blood Flow

Fluorescent microspheres for regional perfusion of myocardium were assessed in tissue obtained at terminal surgery. Myocardial flow was quantified with 15 μm fluorescently labeled microspheres injected into the LV at baseline and during 5-minute infusions of dobutamine at 5 μg/kg/min, with reference arterial blood samples collected under each experimental condition. 40,000 fluorescently labeled microspheres (Triton Technology Inc, San Diego Calif.) per kilogram were injected for each analysis. At study completion, the heart was dissected free and removed en-bloc. The heart was serially sectioned, and samples of fresh tissue rapidly excised from subendocardial and subepicardial samples from the LAD and remote territories for regional perfusion analyses. These tissue samples were then digested, and fluorescent microspheres counted to calculate regional perfusion.

viii. Isolation of Bone Marrow Derived Mesenchymal Stem Cells

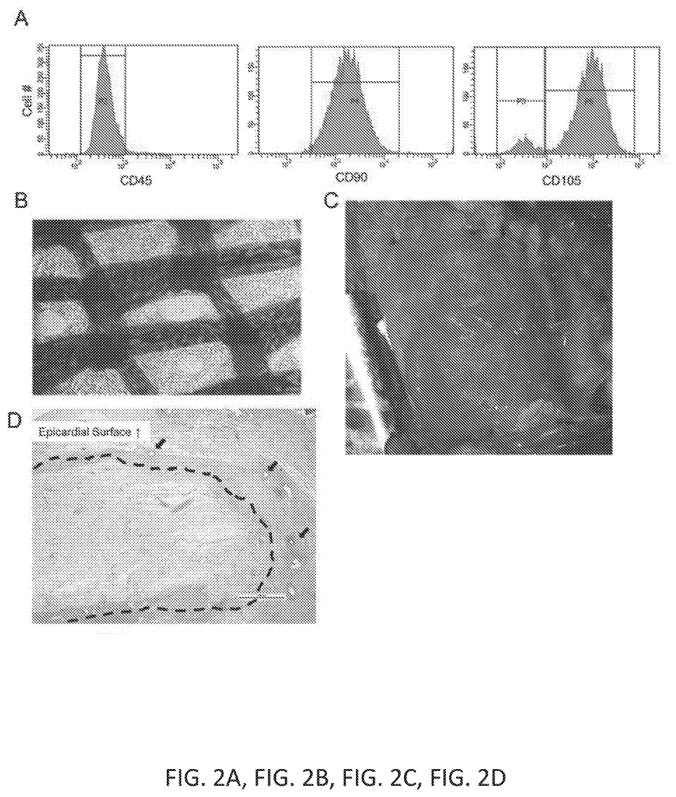

Sterile bone marrow was obtained from swine sternums, collected into a BD Vacutainer CPT tube, then centrifuged for 30 minutes at 1,800×g with no brake to create a density gradient. The buffy coat containing mononuclear cells was removed and washed with HBSS. Mononuclear cells were pelleted by centrifugation and resuspended in growth media (ADMEM with 10% FBS). Mononuclear cells were transferred to a cell culture flask for adherent growth, and MSCs isolated from the mononuclear fraction by their adherent nature. All non-MSCs were washed off within 24 hours, leaving a monolayer of MSCs in the tissue culture flask. MSC phenotype was confirmed by flow cytometry, ensuring negativity for CD45, a hematopoietic marker, and positivity for CD90 and CD105, markers of MSCs (FIG. 2A). Following isolation and culture as described, MSCs seeded onto the patch were 98% viable as measured by trypan blue exclusion and the purity of the cell population was approximately 95% as measured by flow cytometry markers as outlined above.

ix. Creation of Allogeneic MSC Patch

4×10⁶ MSCs were seeded on a sterile polyglactin patch (3.5 cm diameter round) and incubated for 7-10 days until confluent. The MSC-seeded patch was maintained in a sterile environment, washed with sterile PBS, and ultimately sutured onto the epicardium of the anterior heart in the LAD distribution (FIG. 2B) during CABG. The final patch holds 8×10⁶ MSCs.

x. Histology

During terminal surgery, myocardium samples from ischemic and non-ischemic regions of the LV were immediately rinsed and fixed in neutral buffered formalin. Fixed tissue was embedded in paraffin and processed for H&E and trichrome staining for analysis of overall tissue structure, viability, and collagen deposits. Inflammatory cells (neutrophils, eosinophils, lymphocytes, macrophages) were identified through the use of the H&E stain. Different cell types were first identified as cells that had migrated into the tissue and were further identified by criteria such as presence or absence of cytoplasmic granules, intensity of staining of any cytoplasmic granules, nuclear shape (lobulated or non-lobulated), and proportion of cytoplasm to nucleus.

xi. Electron Microscopy

Cardiac tissue samples were immediately isolated and placed in fixative (3% paraformaldehyde, 1.5% glutaraldehyde, and 2.5% sucrose in 0.1 M sodium cacodylate buffer with 5 mM calcium chloride and 5 mM magnesium chloride, pH 7.4) for 12 hours prior to processing for electron microscopy.

Samples approximately 2 mm³ were stored in a fixative solution of 3% paraformaldehyde, 1.5% glutaraldehyde, and 2.5% sucrose in 0.1 M sodium cacodylate buffer with 5 mM calcium chloride and 5 mM magnesium chloride (pH 7.4) for at least 12 hours, rinsed in buffer, then placed in 1% osmium tetroxide and 0.1 M sodium cacodylate buffer for at least 4 hours. Specimens were rinsed in ultrapure water (NANOpure Infinity®; Barnstead/Thermo Fisher Scientific; Waltham, Md.), en bloc stained with 2% aqueous uranyl acetate for 2 hours and rinsed in ultrapure water. They were then dehydrated in an ethanol series (25%, 50%, 75%, 95% (2×) and 100% (3×); 15 min for each change) and embedded in Embed 812 resin (Electron Microscopy Sciences, Hatfield, Pa.). Ultrathin sections 80-100 nm thick were cut on a Leica Ultracut UCT microtome using a diamond knife, collected on formvar/carbon-coated 200-mesh copper grids (Electron Microscopy Sciences, Hatfield, Pa.), and stained with 3% uranyl acetate for 20 min, followed by Sato's triple-lead stain (1) for 3 min. Sections were examined with an FEI Philips CM 12 transmission electron microscope operating at 60 kV. Images were recorded with a Maxim DL digital capture system

xii. Isolation of Nuclear and Mitochondrial Fractions from Cardiac Tissue:

Mitochondrial fractions were isolated from cardiac tissue using commercially available kits (Miltenyi Biotec) and homogenized using the gentleMACS homogenizer (Miltenyi Biotec). Mitochondria were then tagged with magnetic beads conjugated to TOM22, allowing for magnetic separation and isolation of mitochondria. This method of isolation results in 95% purity and does not result in contamination from endoplasmic reticulum. Once isolated, mitochondria were then suspended in RIPA buffer with 1× protease inhibitor for downstream analysis. Nuclear fractions were isolated using commercially available kits (Pierce Thermo Scientific) as described in the manufacturer's protocol. Both mitochondrial and nuclear protein concentrations were determined using a standard BCA assay.

xiii. Western Blot

Western blot was used to identify and quantify the proteins PGC1α in isolated nuclear fractions, and succinate dehydrogenase, and ATP synthase from isolated mitochondrial fractions from HM tissue.

xiv. Proteomic Analysis with TMT™

Identification and relative quantification of mitochondrial proteins isolated from the swine were performed using TMT™ (tandem mass tag) reagents (ThermoFisher, Waltham, Mass.) in conjunction with liquid chromatography and tandem mass spectrometry (LC-MS/MS). The TMT™ isobaric reagent labels all primary amines to yield labeled peptides that are identical in mass and are also identical in single MS mode. Comparisons were made between the protein concentrations from mitochondria of the LAD region from revascularized hibernating pigs (either MSC patch or sham groups) to a normal control in two 10-plex studies using the same normal control in both studies. To label mitochondrial proteins from individual samples, isolates are centrifuged and 40 μg of protein from each sample are rehydrated in 0.5 M triethylammonium bicarbonate buffer, pH 8.5, denatured, reduced, alkylated, trypsin digested independently in parallel and labeled with TMT™ reagents. After labeling the peptides, all samples are pooled and dried in vacuo prior to liquid chromatography and tandem MS. To reduce the complexity of the tryptic peptides, peptides are separated by a strong cation exchange into 16 fractions. The proteins from each fraction are separated by reversed phase high performance liquid chromatography (HPLC) and then introduced (on-line) into a mass spectrometer. The capillary HPLC system is interfaced with an Orbitrap Fusion mass spectrometer (ThermoFisher) via a nano-electrospray ionization source. Protein identification and relative quantification were carried out using Proteome Discoverer 2.1 (ThermoFisher) and Scaffold 4.7.3239 (Proteome Software, Portland, Oreg.) software programs. MS/MS data were searched against a suitable reference protein species-specific sequence database NCBI (www.ncbi.nlm.nih.gov) or UniProt (uniprot.org) plus the common contaminants protein sequences. False discovery rates for protein, peptide and spectral matches were estimated in Scaffold. The average protein relative quantification was calculated from the TMT reporter ions for each reagent pair. This relative quantification is based on the ratio of the mitochondrial protein abundance from the LAD region in each animal compared to the mitochondrial protein abundance from the LAD region of a SHAM animal. This ratio indicates whether protein relative abundance is increased or decreased. The method in Scaffold for normalization of mass spec intensities across sample categories was previously published and the method for reporting statistical differences in protein abundances between sample categories is permutation testing, to which multiple hypothesis testing corrections can be applied.

xv. Statistics

Comparisons of two groups with equal variance were analyzed using an unpaired, two-sided t-test. Comparisons of two groups with unequal variance were analyzed using Mann-Whitney. Data sets were checked for unequal variances using the f-test, and if found unequal, non-parametric tests were used. Comparisons of more than two groups were assessed for equal variance using the Brown-Forsythe and Bartlett's test, then analyzed using ANOVA with Tukey's correction for multiple comparisons. P-values less than 0.05 were considered significant. Tukey's post-hoc test was used to determine significance between groups in instances of multiple comparisons. Statistics were calculated using GraphPad PRISM software (GraphPad Software, Inc., La Jolla, Calif.).

### 2. Results

i. Characterization of an MSC Patch

Histopathology of HM tissue 4 weeks following patch treatment shows that the host myocardium does not react to the patch material with an inflammatory response (FIG. 2C), though some minor inflammation is evident around the suture sites as indicated by staining for inflammatory cells infiltrating into the myocardium.

ii. CABG with MSC patch improves regional cardiac function under inotropic stimulation.

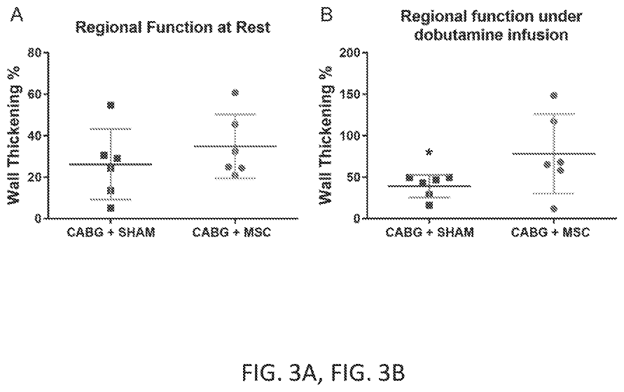

MRI measurements of regional function within the HM region show that CABG+MSC does not alter function at rest compared to CABG+sham (26.24±6.9% vs 34.87±6.3%) (p=0.19) within the ischemic area. However, CABG+MSC significantly improves regional function during inotropic stimulation with low-dose dobutamine (78.24±19.6%) compared to CABG+sham (39.17±5.57%) (p<0.05) (FIG. 3A-B). Cardiac MRI confirmed the absence of necrosis as well as patency of the LIMA-LAD graft distal to the stenosis.

iii. MSC Patch treatment does not significantly alter perfusion in HM under dobutamine challenge.

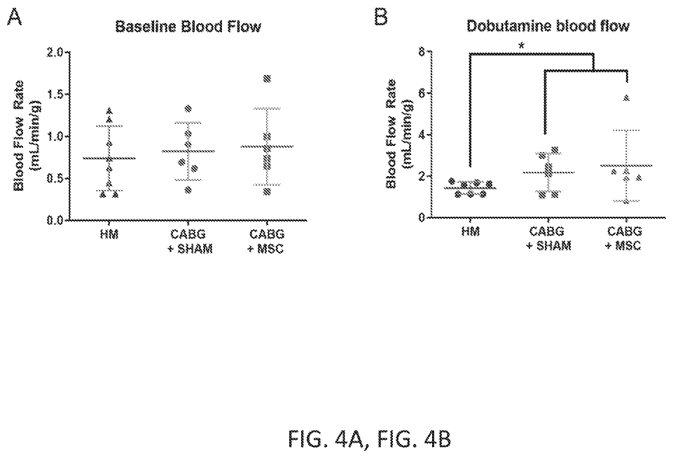

During infusion of low-dose dobutamine, an increase in regional perfusion was noted in revascularized animals (2.18±0.37) compared to HM (1.4±0.11) (p<0.05), with a no significant additional benefit to perfusion in CABG+MSC animals (2.49±0.5) (FIG. 4B). However, measurements of regional perfusion during terminal surgery shows no change at rest (FIG. 4A).

iv. Treatment with an MSC patch improves mitochondrial morphology.

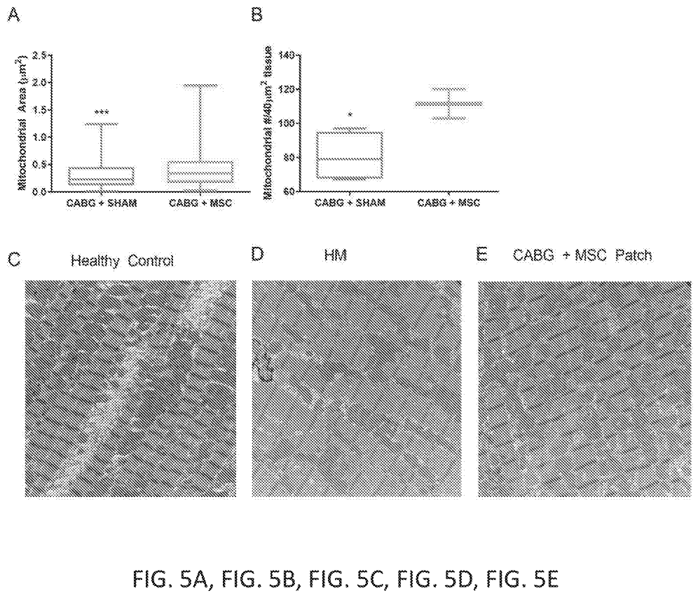

Mitochondrial size (p<0.001) and number (p<0.05) (FIGS. 5A-B) are increased in CABG+MSC compared to CABG+sham as measured by TEM. Representative TEM images (8800× magnification) of HM show dysregulation of mitochondrial size and alignment compared to healthy myocardium (FIGS. 5C-D). CABG+MSC specimens show mitochondrial morphology more consistent with healthy myocardium (FIG. 5E).

v. CABG+MSC patch increases expression of PGC1a, ATP Synthase, and Succinate Dehydrogenase.

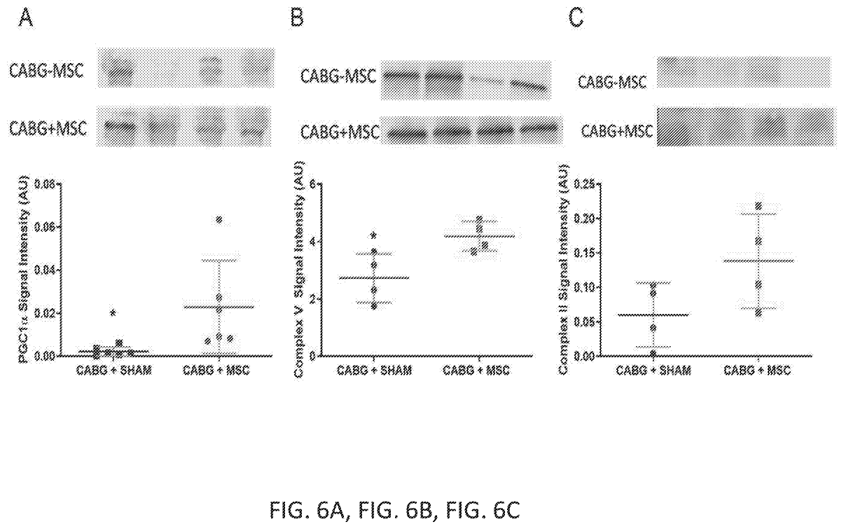

Western blots of CABG+MSC myocardial tissue show a dramatic increase in PGC1α (0.0022±0.0009 vs 0.023±0.009) (p<0.005) along with key components of the ETC including succinate dehydrogenase (complex II) (0.06±0.02 vs 0.14±0.03) (p<0.05) and ATP synthase (complex V) (2.7±0.4 vs 4.2±0.26) (p<0.05) (FIGS. 6A-C). These data indicate that the increase in ETC protein expression may be due to enhanced mitochondrial biogenesis driven by PGC1α signaling.

vi. Proteomic Analysis of HM tissue treated with MSCs indicates increased metabolic function.

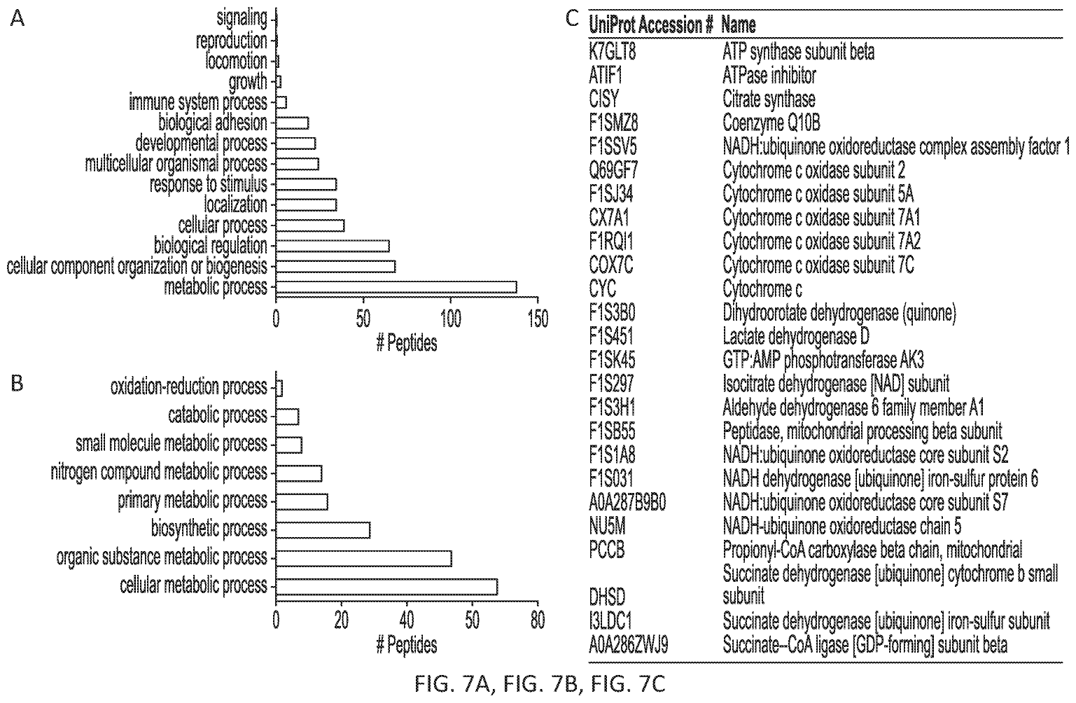

Cardiac mitochondria from both groups were isolated for TMT proteomic profiling. Protein pathway analysis using the PANTHER database of all peptides that showed increased expression following MSC treatment indicate that the increased proteins were predominantly part of the metabolic process (FIG. 7A), and within that pathway, part of the cellular metabolic process (FIG. 7B). The upregulated proteins of interest pertain to the ETC and ATP synthesis (FIG. 7C).

vii. MSC patch therapy for HM results in increased antioxidant signaling.

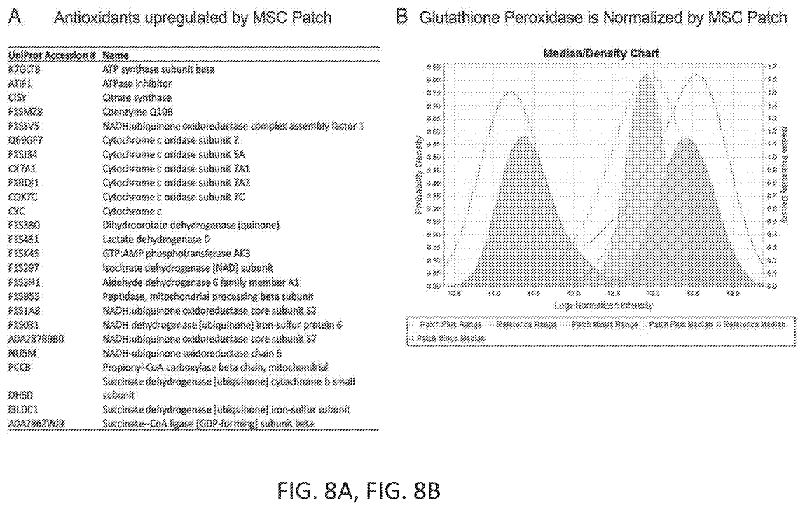

Analysis of antioxidant peptides using TMT proteomics revealed increased antioxidant signaling in CABG+MSC compared to CABG+sham (FIG. 8A), most notably with a 33-fold increase in expression of glutathione peroxidase, a persistent marker of oxidative stress (FIG. 8B). These results indicate that MSC patch therapy may enhance HM recovery through antioxidant pathways and increased clearing of ROS.

### 3. Discussion

Adjunctive treatment with an allogeneic MSC patch during CABG leads to improved regional function in response to inotropic stimulation. This swine model of HM was previously developed and characterized which recreates the clinical experience including reduced regional perfusion and function but with preserved viability as measured by increased glucose uptake. Swine provide an ideal large animal model of CAD as they do not have epicardial bridging collaterals, allowing stenosis of the LAD alone to result in regional ischemia. This model reflects what is seen clinically, including the response to CABG showing improved survival and partial functional recovery. Prior to revascularization, swine with single-vessel stenosis HM do not have detectable impairment in global function as measured by ejection fraction but do exhibit significant reduction in regional wall thickening. As recovery of HM following CABG can result in improved outcome at the three-month interval in humans, recovery has been studied in this model at both one- and three-months following CABG. CMR done at these timepoints demonstrate similar preserved viability and graft patency but persistent regional dysfunction. As benefits were comparable at both time-points, focus is on a time-point of one month following revascularization.

In this study, MSC therapy improved the expression of key ETC and antioxidant proteins. The results support the findings of other investigators that enhanced mitochondrial biogenesis from MSC therapy occurs by enhanced PGC1α signaling. This indicates that MSCs provide a benefit beyond revascularization alone, and sham patch data indicate that the therapeutic effect is not due to structural support from the patch itself. These studies indicate that, predictably, both groups show improvement in regional perfusion after CABG, but adding MSCs does not significantly improve perfusion. This indicates that the mechanism of MSC action can be independent of coronary blood flow.

Bone marrow derived stem cells are among the most commonly researched adult stem cell types. They have been identified as potential therapeutic agents due to their endogenous characteristics including their ability to home to sites of injury and their low immunogenicity due to a lack of expression of major histocompatibility complex II (MHC II) which allows the use of allogeneic cell lines to minimize variability between treatments. Previous studies of MSCs in cardiac disease have primarily focused on models of myocardial infarction (MI), which showed limited therapeutic potential as MSCs are unable to alter infarcted tissue. However, benefits have been shown in the pen-infarct regions of the myocardium. Several studies using stem cell-based therapies have shown increased vascularization, reduced apoptosis, and influx of growth factors which stimulate the endogenous tissue in pen-infarct regions that are injured but not infarcted. HM, much like pen-infarct myocardium, has potential for recovery with MSC treatment. This key observation indicates the therapeutic potential for ischemic but viable tissue seen in HM.

Stem cell administration technique is critical to efficacy of treatment, but the ideal route of delivery remains speculative. Methods include intracoronary injection, direct myocardial injection, and placement of a cell-laden patch on the epicardium. Intracoronary injection is the least invasive as it can be performed percutaneously, and provides distribution throughout the myocardium. However, injected cells are rapidly dispersed by circulation, resulting in low cell retention in the target myocardium. Alternatively, direct myocardial injection allows for targeting specific regions of injury and greater retention of administered cells. A critical limitation of both methods is that up to 95% of stem cells become nonviable during injection or shortly after, and very few are observed in the host tissue at later time points. As a third option, absorbable epicardial patches mixed with cardiac stem cells have resulted in contractile myocardium. Studies of MSCs loaded onto absorbable patches showed improved retention, cardiac function, enhanced angiogenesis and decreased fibrosis 4 weeks following MI. Following these principles, an epicardial stem cell patch was used in order to maximize viability and retention of stem cells.

Despite the wealth of research in stem cell therapy for cardiac disease, there is no unified theory for the mechanism by which stem cells improve cardiac outcome. The original theory of stem cell therapeutics is that administered stem cells migrate to the site of injury and replace injured endogenous cells, engrafting and differentiating into mature cardiomyocytes. However, few studies have been able to conclusively show that this is the case. Regardless of the method of administration, very few administered cells remain present in host tissue following treatment, though functional and physiological benefits are still observed in both animal models and human trials. These findings have shifted the prevailing theory of the mechanism of MSC therapy from cell replacement to that of an unknown paracrine action. In vitro studies of ischemia have shown that the therapeutic effect of MSC co-culture is maintained by treatment with conditioned media alone, supporting the theory of a paracrine mechanism.

Previous work has shown that HM is characterized by reduced expression of mitochondrial ETC proteins, which persists despite revascularization with CABG alone. This study shows that animals receiving MSC treatment show dramatic increases in expression of nuclear-bound PGC1α, a master regulator of mitochondrial biogenesis. Additionally, these animals show significant increases in protein expression of ATP synthase and succinate dehydrogenase—components of the ETC which contribute to mitochondrial respiration and ATP production. These observations indicate that the mechanism by which MSCs improve regional cardiac function is mitochondrial-based and provides a therapeutic benefit beyond that of revascularization alone.

### 4. Conclusion

Clinical and experimental evidence indicates that contractile function in patients with HM will not normalize with CABG alone. The data support the utility of MSC patch as adjunctive therapy at the time of CABG, which results in improved regional function in HM. The study indicates that this observed improvement in function can be due to improved mitochondrial biogenesis and function, as measured by increased PGC1α expression and mitochondrial density. The downstream effects of enhanced mitochondrial biogenesis include increased ETC proteins and improved antioxidant status. These results indicate a mitochondrial mechanism of MSC therapy that translates into the improved contractility seen via MRI.

This pre-clinical stem cell patch has significant clinical implications. It is an adjuvant therapy that can be easily placed at the time of CABG in cardiac patients to improve functional recovery by enhancing endogenous mitochondrial biogenesis. This allows a recovering heart to better respond to increased work, as evidenced by improved contractility under dobutamine infusion. To bridge the gap from bench to bedside, future studies must focus on developing an “off-the-shelf” MSC patch that can be proven safe in humans and broadly utilized for cardiac applications.

### B. Example 2: Mesenchymal Stem Cell Cultivation and Patch Formation Procedure

Objective: Create an MSC-laden patch using surgical mesh scaffolding. This patch will provide adjuvant treatment for Hibernating Myocardium that can be applied to the surface of the heart at the time of revascularization. The mesh scaffold is bio-absorbable and non-reactive.

Allogeneic MSCs can be used as they are immune-privileged and do not cause rejection. This can been confirmed in several clinical trials.

1. Obtain MSCs from Bone Marrow Sample
i. Obtain 2-4 CPT (BD Vacutainer #362753) tubes containing bone marrow.
ii. Centrifuge at 1,800 rcf for 30 minutes at 18° C. with brake deceleration at 0.
iii. Leave CPT tubes at RT, and centrifuge within 2 hours following collection
iv. Fill a 50 ml conical tube with 40 ml of HBSS (Gibco #14175).
v. Remove plasma (top) layer and discard.
vi. Remove buffy coat containing mononuclear cells and add to conical tube containing HBSS. Invert to mix. Each CPT tube should yield approximately 1-2 ml of mononuclear cells
vii. Centrifuge cells at 300×g for 15 minutes.
viii. Remove supernatant and resuspend pellet in 10 mL of supplemented growth media by pipette mixing 20 times.
ix. Count mononuclear cells using a hemocytometer.
x. Transfer cells to a T75 cell culture flask for adherent cell growth.

2. Culture MSCs
i. The following day, remove media and wash cells 3× with 3 mL sterile PBS (Gibco #10010) per wash. Add 8 ml of fresh pre-warmed complete growth media.
   a. During p0, wash cells with sterile 1×PBS prior to feeding to reduce the chance of fungus growth.
   b. Complete Growth Media:
      (A) ADMEM (Gibco #12491)
      (B) 10% FBS (Hyclone #SH30070)
      (C) 1× Glutamax (Invitrogen #35050-061)
      (D) 1× Pen/Strep (Gibco #15140-122)
ii. Check cells under the microscope daily. Small clusters of spindle-shaped MSC's will be visible in about 2 days and will be confluent in 7-10 days when—35×10⁶ mononuclear cells are plated.

Note: The rate of MSC growth is dependent on cell density. The greater the cell density, the faster they grow. If MSC density is low, they will grow slowly.
iii. Feed cells every 2-3 days.
iv. Subculture at 70-80% confluency—split 1:3 or 1:4
v. Use TrypLE (Gibco #A1217702) for cell detachment
vi. Label all plates/flasks with cell type, passage, date and initials

3. Confirm MSC Phenotype by Flow Cytometry
i. Sterilize surgical mesh
ii. Ethylene Oxide gas sterilization 2 days prior to use
iii. Ethicon Vicryl Knitted Mesh (#VKML)
iv. 3.5 cm diameter

4. Seed Mesh with MSCs
i. Seed each patch with 2 million MSCs by pipetting cell slurry in growth media into cell culture dish containing the sterilized surgical mesh
ii. Grow to full confluence (1 week) feeding the patch with fresh growth media every other day

By day 5, MSCs will begin to adhere to the mesh and form a cell sheet with mesh scaffold

5. Prepare for Surgical Implantation
i. In sterile cell culture hood, aspirate growth media off of the cell patch
ii. Rinse 3× with sterile saline
iii. Maintain in sterile conditions until application

6. Surgical Implantation
i. Surgeon will place stem cell patch onto the epicardium and secure with sutures

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Rahimtoola S H. The hibernating myocardium. American heart journal. 1989; 117(1):211-21. PubMed PMID: 2783527.
2. Velazquez E J, Lee K L, Deja M A, Jain A, Sopko G, Marchenko A, Ali I S, Pohost G, Gradinac S, Abraham W T. Coronary-artery bypass surgery in patients with left ventricular dysfunction. New England Journal of Medicine. 2011; 364(17):1607-16.
3. Canty J M, Fallavollita J A. Hibernating myocardium. Journal of nuclear cardiology. 2005; 12(1):104-19.
4. Chen H, Chan D C. Physiological functions of mitochondrial fusion. Annals of the New York Academy of Sciences. 2010; 1201:21-5. Epub 2010/07/24. doi: 10.1111/j.1749-6632.2010.05615.x. PubMed PMID: 20649534.
5. Vidoni S, Zanna C, Rugolo M, Sarzi E, Lenaers G. Why mitochondria must fuse to maintain their genome integrity. Antioxid Redox Signal. 2013; 19:1-10.
6. Gustafsson A, Gottlieb R. Autophagy in Ischemic Heart Disease. Circ Res. 2009; 104:150-8.
7. Galloway C, Lee H, Yoon Y. Mitochondrial morphology-emerging role in bioenergetics. Free Rad Biol & Med. 2012; 53:2218-28.
8. Cabrera J A, Ziemba E A, Colbert R, Anderson L B, Sluiter W, Duncker D J, Butterick T A, Sikora J, Ward H B, Kelly R F, McFalls E O. Altered expression of mitochondrial electron transport chain proteins and improved myocardial energetic state during late ischemic preconditioning. American journal of physiology Heart and circulatory physiology. 2012; 302(10):H1974-82. Epub 2012/03/06. doi: 10.1152/ajpheart.00372.2011. PubMed PMID: 22389388; PMCID: PMC3362109.
9. Holley C T, Long E K, Butterick T A, Duffy C M, Lindsey M E, Stone L H, McFalls E O, Kelly R F. Mitochondrial fusion proteins in revascularized hibernating hearts. Journal of Surgical Research. 2015; 195(1):29-36. doi: 10.1016/j.jss.2014.12.052. PubMed PMID: 25655995.
10. Chen Y, Liu Y, Dorn G. Mitochondrial fusion is essential for oranelle function and cardiac homeostasis. Circ Res. 2011; 109:1327-31.
11. Hocum Stone L L, Swingen C, Holley C, Wright C, Chappuis E, Ward H B, McFalls E O, Kelly R F. Magnetic resonance imaging assessment of cardiac function in a swine model of hibernating myocardium 3 months following bypass surgery. J Thorac Cardiovasc Surg. 2017; 153(3):582-90. Epub 2016/12/13. doi: 10.1016/j.jtcvs.2016.10.089. PubMed PMID: 27939502.
12. Wassef M A E, Tork O M, Rashed L A, Ibrahim W, Morsi H, Rabie D M M. Mitochondrial Dysfunction in Diabetic Cardiomyopathy: Effect of Mesenchymal Stem Cell with PPAR-gamma Agonist or Exendin-4. Experimental and clinical endocrinology & diabetes: official journal, German Society of Endocrinology [and] German Diabetes Association. 2018; 126(1):27-38. Epub 2017/04/28. doi: 10.1055/s-0043-106859. PubMed PMID: 28449155.
13. Perico L, Morigi M, Rota C, Breno M, Mele C, Noris M, Introna M, Capelli C, Longaretti L, Rottoli D, Conti S, Coma D, Remuzzi G, Benigni A. Human mesenchymal stromal cells transplanted into mice stimulate renal tubular cells and enhance mitochondrial function. Nature communications. 2017; 8(1):983. Epub 2017/10/19. doi: 10.1038/s41467-017-00937-2. PubMed PMID: 29042548; PMCID: PMC5754365.
14. Hocum Stone L, Wright C, Chappuis E, Messer M, Ward H B, McFalls E O, Kelly R F. Surgical Swine Model of Chronic Cardiac Ischemia Treated by Off-Pump Coronary Artery Bypass Graft Surgery. J Vis Exp. 2018(133). Epub 2018/04/17. doi: 10.3791/57229. PubMed PMID: 29658941; PMCID: PMC5933266.
15. Pittenger M F, Martin B J. Mesenchymal stem cells and their potential as cardiac therapeutics. Circulation research. 2004; 95(1):9-20.
16. Muruganuj an A, Mills C, Kang D, Tang H, Huang X, Mi H, Thomas P D. PANTHER version 11: expanded annotation data from Gene Ontology and Reactome pathways, and data analysis tool enhancements. Nucleic acids research. 2016; 45(D1):D183-D9. doi: 10.1093/nar/gkw1138.
17. Mi H, Thomas P. PANTHER Pathway: An Ontology-Based Pathway Database Coupled with Data Analysis Tools. In: Nikolsky Y, Bryant J, editors. Protein Networks and Pathway Analysis. Totowa, N.J.: Humana Press; 2009. p. 123-40.
18. Kelly R F, Cabrera J A, Ziemba E A, Crampton M, Anderson L B, McFalls E O, Ward H B. Continued depression of maximal oxygen consumption and mitochondrial proteomic expression despite successful coronary artery bypass grafting in a swine model of hibernation. The Journal of thoracic and cardiovascular surgery. 2011; 141(1):261-8. doi: 10.1016/j.jtcvs.2010.08.061. PubMed PMID: 21168030.
19. Cabrera J A, Butterick T A, Long E K, Ziemba E A, Anderson L B, Duffy C M, Sluiter W, Duncker D J, Zhang J, Chen Y, Ward H B, Kelly R F, McFalls E O. Reduced expression of mitochondrial electron transport chain proteins from hibernating hearts relative to ischemic preconditioned hearts in the second window of protection. Journal of molecular and cellular cardiology. 2013; 60:90-6. Epub 2013/04/09. doi: 10.1016/j.yjmcc.2013.03.018. PubMed PMID: 23562790.
20. Cabrera J A, Ziemba E A, Colbert R, Kelly R F, Kuskowski M, Arriaga E A, Sluiter W, Duncker D J, Ward H B, McFalls E O. Uncoupling protein-2 expression and effects on mitochondrial membrane potential and oxidant stress in heart tissue. Translational research: the journal of laboratory and clinical medicine. 2012; 159(5):383-90. Epub 2012/04/17. doi: 10.1016/j.trs1.2011.11.001. PubMed PMID: 22500511; PMCID: 3328031.
21. McFalls E, Baldwin D, Palmer B, Marx D, Jaimes D, Ward H. Regional glucose uptake within hypoperfused swine myocardium as measured by positron emission tomography. American Journal of Physiology-Heart and Circulatory Physiology. 1997; 272(1):H343-H9.
22. White F, Carroll S, Magnet A, Bloor C. Coronary collateral development in swine after coronary artery occlusion. Circulation Research. 1992; 71(6):1490-500.
23. Holley C T, Long E K, Lindsey M E, McFalls E O, Kelly R F. Recovery of Hibernating Myocardium: What Is the Role of Surgical Revascularization? Journal of Cardiac Surgery. 2015; 30(2):224-31. doi: doi:10.1111/jocs.12477. PubMed PMID: 25470424; PMCID: 25470424
24. Shah B N, Khattar R S, Senior R. The hibernating myocardium: current concepts, diagnostic dilemmas, and clinical challenges in the post-STICH era. European heart journal. 2013; 34(18):1323-36. doi: 10.1093/eurheartj/eht018. PubMed PMID: 23420867.
25. Hughey C C, James F D, Ma L, Bracy D P, Wang Z, Wasserman D H, Rottman J N, Shearer J. Diminishing impairments in glucose uptake, mitochondrial content, and ADP-stimulated oxygen flux by mesenchymal stem cell therapy in the infarcted heart. Am J Physiol Cell Physiol. 2014; 306(1):C19-27. doi: 10.1152/ajpce11.00156.2013. PubMed PMID: 24196528; PMCID: PMC3919976.
26. Shake J G, Gruber P J, Baumgartner W A, Senechal G, Meyers J, Redmond J M, Pittenger M F, Martin B J. Mesenchymal stem cell implantation in a swine myocardial infarct model: engraftment and functional effects. The Annals of thoracic surgery. 2002; 73(6):1919-26.
27. Discher D E, Mooney D J, Zandstra P W. Growth Factors, Matrices, and Forces Combine and Control Stem Cells. Science. 2009; 324(5935):1673-7. doi: 10.1126/science.1171643.
28. Aggarwal S, Pittenger M F. Human mesenchymal stem cells modulate allogeneic immune cell responses. Blood. 2005; 105(4):1815-22.
29. Ankrum J A, Ong J F, Karp J M. Mesenchymal stem cells: immune evasive, not immune privileged. Nature biotechnology. 2014; 32(3):252-60.
30. Keating A. Mesenchymal stromal cells: new directions. Cell stem cell. 2012; 10(6):709-16.
31. Hare J M, Fishman J E, Gerstenblith G, DiFede Velazquez D L, Zambrano J P, Suncion V Y, Tracy M, Ghersin E, Johnston P V, Brinker J A, Breton E, Davis-Sproul J, Schulman I H, Byrnes J, Mendizabal A M, Lowery M H, Rouy D, Altman P, Wong Po Foo C, Ruiz P, Amador A, Da Silva J, McNiece I K, Heldman A W, George R, Lardo A. Comparison of allogeneic vs autologous bone marrow-derived mesenchymal stem cells delivered by transendocardial injection in patients with ischemic cardiomyopathy: the POSEIDON randomized trial. Jama. 2012; 308(22):2369-79. Epub 2012/11/03. doi: 10.1001/jama.2012.25321. PubMed PMID: 23117550; PMCID: PMC4762261.
32. Marbán E, Malliaras K. Mixed results for bone marrow-derived cell therapy for ischemic heart disease. Jama. 2012; 308(22):2405-6.
33. Hong K U, Bolli R. Cardiac stem cell therapy for cardiac repair. Current treatment options in cardiovascular medicine. 2014; 16(7):1-19.
34. Siltanen A, Nuutila K, Imanishi Y, Uenaka H, Makela J, Patila T, Vento A, Miyagawa S, Sawa Y, Harjula A. The paracrine effect of skeletal myoblasts is cardioprotective against oxidative stress and involves EGFR-ErbB4 signaling, cystathionase, and the unfolded protein response. Cell transplantation. 2016; 25(1):55-69.
35. Suzuki G, Iyer V, Lee T-C, Canty J M. Autologous mesenchymal stem cells mobilize cKit+ and CD133+ bone marrow progenitor cells and improve regional function in hibernating myocardium. Circulation research. 2011:CIRCRESAHA. 111.245969.
36. Ranganath S H, Levy O, Inamdar M S, Karp J M. Harnessing the mesenchymal stem cell secretome for the treatment of cardiovascular disease. Cell stem cell. 2012; 10(3):244-58.
37. Malliaras K, Makkar R R, Smith R R, Cheng K, Wu E, Bonow R O, Marban L, Mendizabal A, Cingolani E, Johnston P V. Intracoronary cardiosphere-derived cells after myocardial infarction: evidence of therapeutic regeneration in the final 1-year results of the CADUCEUS trial (CArdiosphere-Derived aUtologous stem CElls to reverse ventricUlar dySfunction). Journal of the American College of Cardiology. 2014; 63(2):110-22.
38. Hofmann M, Wollert K C, Meyer G P, Menke A, Arseniev L, Hertenstein B, Ganser A, Knapp W H, Drexler H. Monitoring of bone marrow cell homing into the infarcted human myocardium. Circulation. 2005; 111(17):2198-202.
39. Kaminski A, Steinhoff G, editors. Current status of intramyocardial bone marrow stem cell transplantation. Seminars in thoracic and cardiovascular surgery; 2008: Elsevier.
40. Zimmermann W-H, Eschenhagen T. Cardiac tissue engineering for replacement therapy. Heart failure reviews. 2003; 8(3):259-69.
41. Zimmermann W-H, Melnychenko I, Wasmeier G, Didie M, Naito H, Nixdorff U, Hess A, Budinsky L, Brune K, Michaelis B. Engineered heart tissue grafts improve systolic and diastolic function in infarcted rat hearts. Nature medicine. 2006; 12(4):452-8.
42. Lee Y S, Lim K S, Oh J-E, Yoon A-R, Joo W S, Kim H S, Yun C-O, Kim S W. Development of porous PLGA/PEI 1.8 k biodegradable microspheres for the delivery of mesenchymal stem cells (MSCs). Journal of Controlled Release. 2015; 205:128-33.
43. Orlic D, Kajstura J, Chimenti S, Jakoniuk I, Anderson S M, Li B, Pickel J, McKay R, Nadal-Ginard B, Bodine D M. Bone marrow cells regenerate infarcted myocardium. Nature. 2001; 410(6829): 701-5.
44. Oh H, Bradfute S B, Gallardo T D, Nakamura T, Gaussin V, Mishina Y, Pocius J, Michael L H, Behringer R R, Garry D J. Cardiac progenitor cells from adult myocardium: homing, differentiation, and fusion after infarction. Proceedings of the National Academy of Sciences. 2003; 100(21):12313-8.
45. Murry C E, Soonpaa M H, Reinecke H, Nakajima H, Nakajima H O, Rubart M, Pasumarthi K B, Virag J I, Bartelmez S H, Poppa V. Haematopoietic stem cells do not transdifferentiate into cardiac myocytes in myocardial infarcts. Nature. 2004; 428(6983):664-8.
46. Nygren J M, Jovinge S, Breitbach M, Säwén P, Röll W, Hescheler J, Taneera J, Fleischmann B K, Jacobsen S E W. Bone marrow-derived hematopoietic cells generate cardiomyocytes at a low frequency through cell fusion, but not transdifferentiation. Nature medicine. 2004; 10(5):494-501.
47. Malliaras K, Li T S, Luthringer D, Terrovitis J, Cheng K, Chakravarty T, Galang G, Zhang Y, Schoenhoff F, Van Eyk J, Marban L, Marban E. Safety and efficacy of allogeneic cell therapy in infarcted rats transplanted with mismatched cardiosphere-derived cells. Circulation. 2012; 125(1):100-12. Epub 2011/11/17. doi: 10.1161/circulationaha.111.042598. PubMed PMID: 22086878; PMCID: PMC3256094.
48. Stamm C, Westphal B, Kleine H-D, Petzsch M, Kittner C, Klinge H, Schümichen C, Nienaber C A, Freund M, Steinhoff G. Autologous bone-marrow stem-cell transplantation for myocardial regeneration. The Lancet. 2003; 361(9351):45-6.
49. Ahmadi H, Baharvand H, Ashtiani S K, Soleimani M, Sadeghian H, Ardekani J M, Mehrjerdi N Z, Kouhkan A, Namiri M, Madani-Civi M. Safety analysis and improved cardiac function following local autologous transplantation of CD133+ enriched bone marrow cells after myocardial infarction. Current neurovascular research. 2007; 4(3):153-60.
50. Zhao Q, Sun Y, Xia L, Chen A, Wang Z. Randomized study of mononuclear bone marrow cell transplantation in patients with coronary surgery. The Annals of thoracic surgery. 2008; 86(6): 1833-40.
51. Timmers L, Lim S K, Hoefer I E, Arslan F, Lai R C, van Oorschot A A, Goumans M J, Strijder C, Sze S K, Choo A. Human mesenchymal stem cell-conditioned medium improves cardiac function following myocardial infarction. Stem cell research. 2011; 6(3):206-14.
Sato T. A modified method for lead staining of thin sections. Journal of electron microscopy. 1968; 17(2):158-9.
Cabrera J A, Ziemba E A, Colbert R, Anderson L B, Sluiter W, Duncker D J, et al. Altered expression of mitochondrial electron transport chain proteins and improved myocardial energetic state during late ischemic preconditioning. American journal of physiology Heart and circulatory physiology. 2012 May 15; 302(10):H1974-82.

We claim:

1. A method of increasing cardiac function in a subject's myocardium comprising: administering a scaffold to the subject's myocardium, wherein the scaffold comprises mesenchymal stem cells (MSCs).

2. The method of claim 1, wherein the subject's heart myocardium is hibernating myocardium.

3. The method of any one of claims 1-2, wherein the scaffold is administered to the subject in combination with coronary artery bypass graft surgery.

4. The method of any one of claims 1-3, wherein the subject has undergone coronary artery bypass graft surgery.

5. The method of any one of claims 1-4, wherein the scaffold is a surgical mesh or any other bioabsorbable woven material.

6. The method of claim 5, wherein the surgical mesh is a polygalactin-based mesh, or any other absorbable woven suture material made primarily of polyglycolic acid.

7. The method of claim 6, wherein the surgical mesh is a bio-absorbable polygalactin surgical mesh.

8. The method of any one of claims 1-7, wherein the MSCs are seeded onto the scaffold prior to administration.

9. The method of any one of claims 1-8, wherein the scaffold comprises a monolayer or multi-layer biofilm of MSCs.

10. The method of any one of claims 1-9, wherein the scaffold comprises 4-10×10⁶ MSCs.

11. The method of any one of claims 1-10, wherein the scaffold is cultured with 4×10⁶ MSCs prior to attaching it to the myocardium.

12. The method of any one of claims 1-11, wherein administering the scaffold comprises attaching the scaffold to the myocardium via suture, surgical clips, staples or glue.

13. The method of any one of claims 1-12, wherein the MSCs are CD90+ and CD105+ and CD45−.

14. The method of any one of claims 1-13, wherein ATP synthase expression is increased in mitochondria present in the myocardium.

15. The method of any one of claims 1-14, wherein succinate dehydrogenase expression is increased in mitochondria present in the myocardium.

16. The method of any one of claims 1-15, wherein PGC1α expression is increased in mitochondria present in the myocardium.

17. A method of restoring the mitochondrial proteome in a subject's myocardium comprising: administering a scaffold to the subject's myocardium, wherein the scaffold comprises mesenchymal stem cells.

18. The method of claim 17, wherein the subject's heart myocardium is hibernating myocardium.

19. The method of any one of claims 17-18, wherein the scaffold is administered to the subject in combination with coronary artery bypass graft surgery.

20. The method of any one of claims 17-19, wherein the subject has undergone coronary artery bypass graft surgery.

21. The method of any one of claims 17-20, wherein the scaffold is a surgical mesh or any other bioabsorbable woven material.

22. The method of claim 21, wherein the surgical mesh is a polygalactin-based mesh, or any other absorbable woven suture material made primarily of polyglycolic acid.

23. The method of claim 22, wherein the surgical mesh is a bio-absorbable polygalactin surgical mesh.

24. The method of any one of claims 17-23, wherein the MSCs are seeded onto the scaffold prior to administration.

25. The method of any one of claims 17-24, wherein the scaffold comprises a monolayer or multi-layer biofilm of MSCs.

26. The method of any one of claims 17-25, wherein the scaffold comprises 4-10×10⁶ MSCs.

27. The method of any one of claims 17-26, wherein the scaffold is cultured with 4×10⁶ MSCs prior to attaching it to the myocardium.

28. The method of any one of claims 17-27, wherein administering the scaffold comprises attaching the scaffold to the myocardium via suture, surgical clips, staples or glue.

29. The method of any one of claims 17-28, wherein the MSCs are CD90+ and CD105+ and CD45−.

30. The method of any one of claims 17-29, wherein ATP synthase expression is increased in mitochondria present in the myocardium.

31. The method of any one of claims 17-30, wherein succinate dehydrogenase expression is increased in mitochondria present in the myocardium.

32. The method of any one of claims 17-31, wherein PGC1α expression is increased in mitochondria present in the myocardium.

33. A method of treating hibernating myocardium comprising combining surgical revascularization with attaching a scaffold comprising mesenchymal stem cells (MSCs) to myocardium.

34. The method of claim 33, wherein the scaffold is administered to the subject in combination with coronary artery bypass graft surgery.

35. The method of any one of claims 33-34, wherein the subject has undergone coronary artery bypass graft surgery.

36. The method of any one of claims 33-35, wherein the scaffold is a surgical mesh or any other bioabsorbable woven material.

37. The method of claim 36, wherein the surgical mesh is a polygalactin-based mesh, or any other absorbable woven suture material made primarily of polyglycolic acid.

38. The method of claim 37, wherein the surgical mesh is a bio-absorbable polygalactin surgical mesh.

39. The method of any one of claims 33-38, wherein the MSCs are seeded onto the scaffold prior to administration.

40. The method of any one of claims 33-39, wherein the scaffold comprises a monolayer or multi-layer biofilm of MSCs.

41. The method of any one of claims 33-40, wherein the scaffold comprises 4-10×10⁶ MSCs.

42. The method of any one of claims 33-41, wherein the scaffold is cultured with 4×10⁶ MSCs prior to attaching it to the myocardium.

43. The method of any one of claims 33-42, wherein administering the scaffold comprises attaching the scaffold to the myocardium via suture, surgical clips, staples or glue.

44. The method of any one of claims 33-43, wherein the MSCs are CD90+ and CD105+ and CD45-.

45. The method of any one of claims 33-44, wherein ATP synthase expression is increased in mitochondria present in the myocardium.

46. The method of any one of claims 33-45, wherein succinate dehydrogenase expression is increased in mitochondria present in the myocardium.

47. The method of any one of claims 33-46, wherein PGC1α expression is increased in mitochondria present in the myocardium.

48. The method of claim 33, wherein surgical revascularization is coronary artery bypass graft surgery or percutaneous coronary intervention.

49. A method of increasing mitochondrial biogenesis in ischemic myocardium comprising administering a scaffold comprising MSCs to the ischemic myocardium.

50. The method of claim 49, wherein the subject's heart myocardium is hibernating myocardium.

51. The method of any one of claims 49-50, wherein the scaffold is administered to the subject in combination with coronary artery bypass graft surgery.

52. The method of any one of claims 49-51, wherein the subject has undergone coronary artery bypass graft surgery.

53. The method of any one of claims 49-52, wherein the scaffold is a surgical mesh or any other bioabsorbable woven material.

54. The method of claim 53, wherein the surgical mesh is a polygalactin-based mesh, or any other absorbable woven suture material made primarily of polyglycolic acid.

55. The method of claim 54, wherein the surgical mesh is a bio-absorbable polygalactin surgical mesh.

56. The method of any one of claims 49-55, wherein the MSCs are seeded onto the scaffold prior to administration.

57. The method of any one of claims 49-56, wherein the scaffold comprises a monolayer or multi-layer biofilm of MSCs.

58. The method of any one of claims 49-57, wherein the scaffold comprises 4-10×10⁶ MSCs.

59. The method of any one of claims 49-58, wherein the scaffold is cultured with 4×10⁶ MSCs prior to attaching it to the myocardium.

60. The method of any one of claims 49-59, wherein administering the scaffold comprises attaching the scaffold to the myocardium via suture, surgical clips, staples or glue.

61. The method of any one of claims 49-60, wherein the MSCs are CD90+ and CD105+ and CD45-.

62. The method of any one of claims 49-61, wherein ATP synthase expression is increased in mitochondria present in the myocardium.

63. The method of any one of claims 49-62, wherein succinate dehydrogenase expression is increased in mitochondria present in the myocardium.

64. The method of any one of claims 49-63, wherein PGC1α expression is increased in mitochondria present in the myocardium.

65. A method of increasing PGC1α in ischemic myocardium comprising administering a scaffold comprising MSCs to the ischemic myocardium.

66. The method of claim 65, wherein the subject's heart myocardium is hibernating myocardium.

67. The method of any one of claims 65-66, wherein the scaffold is administered to the subject in combination with coronary artery bypass graft surgery.

68. The method of any one of claims 65-67, wherein the subject has undergone coronary artery bypass graft surgery.

69. The method of any one of claims 65-68, wherein the scaffold is a surgical mesh or any other bioabsorbable woven material.

70. The method of claim 69, wherein the surgical mesh is a polygalactin-based mesh, or any other absorbable woven suture material made primarily of polyglycolic acid.

71. The method of claim 70, wherein the surgical mesh is a bio-absorbable polygalactin surgical mesh.

72. The method of any one of claims 69-71, wherein the MSCs are seeded onto the scaffold prior to administration.

73. The method of any one of claims 69-72, wherein the scaffold comprises a monolayer or multi-layer biofilm of MSCs.

74. The method of any one of claims 69-73, wherein the scaffold comprises 4-10×10⁶ MSCs.

75. The method of any one of claims 69-74, wherein the scaffold is cultured with 4×10⁶ MSCs prior to attaching it to the myocardium.

76. The method of any one of claims 69-75, wherein administering the scaffold comprises attaching the scaffold to the myocardium via suture, surgical clips, staples or glue.

77. The method of any one of claims 69-76, wherein the MSCs are CD90+ and CD105+ and CD45-.

78. The method of any one of claims 69-77, wherein ATP synthase expression is increased in mitochondria present in the myocardium.

79. The method of any one of claims 69-78, wherein succinate dehydrogenase expression is increased in mitochondria present in the myocardium.

80. The method of any one of claims 69-79, wherein PGC1α expression is increased in mitochondria present in the myocardium.

81. The method of any one of claims 69-80, wherein an increase in PGC1α comprises an increase in PGC1α gene expression and/or protein expression.

82. The method of any one of claims 69-81, wherein the ischemic myocardium is hibernating myocardium.
